(19) United States
(12) Patent Application Publication
(10) Pub. No.: US 2023/0045769 A1
(43) Pub. Date: Feb. 9, 2023

(54) MASS SPECTROMETRY-BASED STRATEGY FOR DETERMINING PRODUCT-RELATED VARIANTS OF A BIOLOGIC

(71) Applicant: Regeneron Pharmaceuticals, Inc., Tarrytown, NY (US)

(72) Inventors: Zhengqi Zhang, Goldens Bridge, NY (US); Yuetian Yan, Chappaqua, NY (US); Shunhai Wang, Scarsdale, NY (US)

(21) Appl. No.: 17/863,303

(22) Filed: Jul. 12, 2022

Related U.S. Application Data
(60) Provisional application No. 63/221,436, filed on Jul. 13, 2021.

(51) Int. Cl. G01N 33/68; G01N 30/72

(52) U.S. Cl. CPC G01N 33/6848; G01N 33/6854; G01N 30/72

(57) ABSTRACT

The present invention relates to the field of protein characterization, and in particular to methods for identifying critical quality attributes of therapeutic proteins expressed in host cells by implementing a workflow including using a competitive binding assay with insufficient antigen followed by SCX-MS.

### CROSS-REFERENCE TO RELATED APPLICATIONS

This application claims priority to and the benefit of U.S. Provisional Patent Application No. 63/221,436, filed Jul. 13, 2021 which is herein incorporated by reference.

### FIELD

The invention generally pertains to methods for determining product-related variants critical for maintaining the structure and function of a biologic using a competitive binding-mass spectrometry workflow.

### BACKGROUND

Biologics have emerged as important drugs for the treatment of cancer, autoimmune disease, infection and cardiometabolic disorders, and they represent one of the fastest growing product segments of the pharmaceutical industry. Biologics must meet very high standards of purity. Thus, it can be important to monitor impurities at different stages of drug development, production, storage and handling. It is often difficult to fully evaluate the impact of the large number of quality attributes that may be related to safety and efficacy. The effects of manufacturing process parameters and material attributes on product quality variations are also difficult to fully characterize.

For robust manufacturing operations, it is important that an integrated control strategy is developed and improved over time based on systematic process characterization along with implementation of appropriate risk assessment and mitigation throughout the product lifecycle. Thus, there is a need for a quality by design standard. The U.N.'s World Health Organization recommends quality by design as a standard because it is harder (and practically impossible) to implement effective quality controls solely by testing a product after the fact. Critical quality attributes (CQAs) serve as the benchmarks that most quality by design implementations revolve around. A CQA is a physical, chemical, biological, or microbiological property or characteristic that should be within an appropriate limit, range, or distribution to ensure the desired product quality. CQAs are generally associated with the drug substance, excipients, intermediates (in-process materials), and drug product”. For biologics, CQAs can be product or process related impurities. Product related impurities can include size variants (aggregates or fragments), variants with post-translational modifications or charge variants. Process related impurities are an inherent part of the process, such as the host cells' DNA or host cell proteins (HCPs), leachables (such as protein A), and viruses. The presence of these impurities in the final drug product can affect product purity, product efficacy and stability.

Identifying CQAs for biologics can, therefore, be a complicated process. Currently, liquid chromatography-tandem mass spectrometry (LC-MS/MS), electrospray ionization-mass spectrometry (ESI-MS), fractionation, or variant identification can be used for physicochemical characterization of intact or digested biologics. Activity characterization can be conducted using ELISA-based bioassays, cell-based bioassays, or surface plasmon resonance (SPR) or biolayer interferometry (BLI) for binding activities. For these methods, product-related CQAs need to be enriched or isolated first and then evaluated individually or based upon experience or prior knowledge. Such an approach to a workflow can result in low throughput.

Thus, there is a long felt need in the art for an efficient method for determining such quality control attributes.

### SUMMARY

Exemplary embodiments disclosed herein satisfy the aforementioned demands by providing methods for identifying product-related CQAs by enriching them.

This disclosure provides for characterizing at least one product-related variant, said method comprising obtaining a sample including a protein of interest and at least one product-related variant of said protein of interest; contacting said sample to a competitive binding condition including an insufficient target immobilized on beads; washing said beads to collect a flow-through; subjecting said flow-through to liquid chromatography-mass spectrometry analysis to separate said protein of interest and said at least one product-related variant; and comparing the abundance of said at least one product-related variant to an abundance of said at least one product-related variant obtained from a liquid chromatography-mass spectrometry analysis of a control sample prior to contacting said sample to said competitive binding condition to characterize said at least one product-related variant.

In one aspect of this embodiment, the target is an antigen against which the protein of interest is directed.

In one aspect of this embodiment, the binding condition provides an insufficient target immobilized on beads. In the same or another aspect of this embodiment, the at least one product-related variant has compromised binding with said insufficient target.

In one aspect of this embodiment, the liquid chromatography is cation-exchange chromatography. In a specific aspect of this embodiment, the liquid chromatography is a strong cation-exchange chromatography.

In one aspect of this embodiment, the mass spectrometer is an electrospray ionization mass spectrometer. In a specific aspect of this embodiment, the mass spectrometer is a nano-electrospray ionization mass spectrometer

In one aspect of this embodiment, said beads are magnetic. In another aspect of this embodiment, said beads are non-magnetic. In a further aspect, said beads are agarose beads. In yet another aspect, said beads are capable of being coated with a peptide or a protein.

In one aspect of this embodiment, wherein said flow-through is enriched for said at least one product-related variant.

In the same or another aspect of this embodiment, said flow-through is collected by performing centrifugation.

In one aspect of this embodiment, said target is biotinylated before immobilizing on said beads. In the same or other aspects of this embodiment, said beads are coated with streptavidin resin. In a specific aspect of this embodiment, said beads are non-magnetic. In another specific aspect, said beads are magnetic.

In one aspect of this embodiment, said insufficient target is such that the amount of said target allows binding of about 30% to about 80% of the protein of interest.

In another aspect of this embodiment, said sample is incubated for about an hour prior to washing. In the same or other aspects of this embodiment, said sample is incubated at room temperature prior to washing.

In one aspect of this embodiment, the method is capable of identifying more than one product-related variant. In a specific aspect, said product-related variant comprises a size-variant. In a specific aspect, said size-variant is a fragmentation variant of said protein of interest. In a specific aspect, said size-variant is an aggregation variant of said protein of interest.

In one aspect of the embodiment, said product-related variant comprises a charge-variant of said protein of interest. In a specific aspect, said product-related variant comprises a post translationally modified-variant of said protein of interest.

In one aspect of this embodiment, said product-related variant is classified as a critical quality attribute if said abundance of said at least product-related variant is significantly more than said abundance of said at least product-related variant in the sample prior to contacting said sample to said competitive binding condition.

### BRIEF DESCRIPTION OF THE DRAWINGS

FIG. 1 is a representation of possible different product-related variants of an antibody including size variants, charge variants, and post-translational modifications (PTMs).

FIG. 2 is a representation of methods routinely used to determine or monitor CQAs during protein drug development.

FIG. 3A shows a method for identifying at least one product-related variant according to an exemplary embodiment.

FIG. 3B shows a method for identifying at least one product-related variant according to an exemplary embodiment.

FIG. 4 shows a method design and workflow for a method for identifying at least one product-related variant according to an exemplary embodiment.

FIG. 5 shows a method design and workflow to determine the antigen to antibody ratio according to an exemplary embodiment.

FIG. 6 shows a titration curve obtained to determine the antigen to antibody ratio according to an exemplary embodiment.

FIG. 7 shows a chromatogram of a sample not enriched for product-related variants of mAb1 according to an exemplary embodiment.

FIG. 8 shows comparison of chromatograms of a sample enriched for product-related variants of mAb1 with reduced binding affinity according to an exemplary embodiment and the control experiment.

FIG. 9 shows comparison of extracted ion chromatograms (XICs) of different product-related variants of mAb1 enriched for product-related variants with reduced binding affinity according to an exemplary embodiment to the control experiment.

FIG. 10 shows a chart of relative percentages of product-related variants of mAb1 identified using a method according to an exemplary embodiment and control experiment.

FIG. 11 shows the structure of bsAb1.

FIG. 12 shows a comparison of XICs of a sample enriched for product-related variants of mAb2 with reduced binding affinity according to an exemplary embodiment and the control experiment.

FIG. 13 shows a chart of relative percentages of the deamidation variant of bsAb1 identified using a method according to an exemplary embodiment and control experiment.

FIG. 14 shows a chart of relative percentages of product-related variants of bsAb1 identified using a method according to an exemplary embodiment and control experiment.

### DETAILED DESCRIPTION

Identification and quantification of product-related variants in biologic products can be very important during the production and development of a product. The identification of such variants can be imperative into developing a safe and effective product. Hence, a robust method and/or workflow to characterize CQAs can be beneficial.

The Annex to ICH Q8 defines CQAs as physical, chemical, biological or microbiological properties or characteristics that should be within an appropriate limit, range or distribution to ensure the desired product quality, safety/immunogenicity, efficacy and pharmacodynamics/pharmacokinetics. (US Food and Drug Administration. Guidance for industry: Q8(R2) pharmaceutical development. www.fda.gov/media/71535/download). Thus, CQAs must be within an appropriate limit, range or distribution to ensure the desired product quality, safety and efficacy. For example, for monoclonal antibody therapeutics that rely on fraction crystalizable (Fc)-mediated effector function for their clinical activity, the terminal sugars of Fc glycans have been shown to be critical for safety or efficacy. Such CQAs include product-related variants, such as size and charge variants, which can impact binding of the protein of interest.

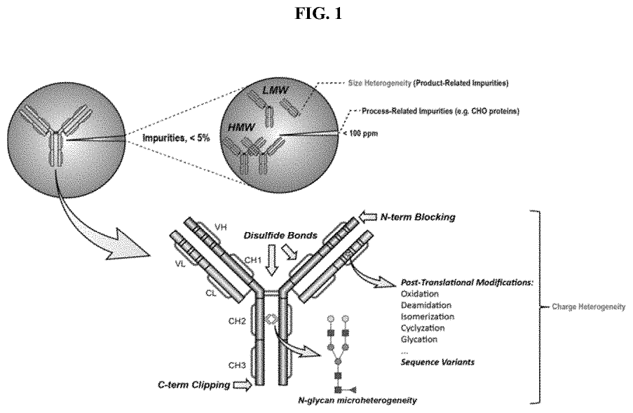

FIG. 1 shows a non-limiting example of variants that can affect the critical quality attribute of a protein. In case of the antibody represented in FIG. 1, the product-related impurities can be size variants like fragmentation products (LMW) and aggregation products (HMW). Other product-related impurities can be charge variants formed due to N-term blocking, disulfide bond formation, C-term clipping, Fc glycan microheterogeneity, or post-translational modifications. These can cause decrease binding of the protein of interest and need to be monitored at various parts of the manufacturing and delivery process.

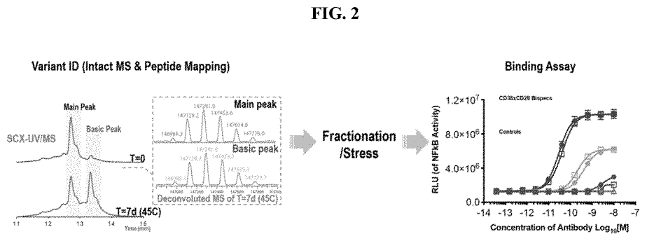

One of the conventional methods includes use of strong cation exchange chromatography (SCX). One such workflow is shown in FIG. 2. This includes performing separation of the protein of interest and its variants by SCX followed by conducting a binding assay of the protein of interest and its variant to identify if the variant has compromised, i.e., reduced binding affinity compared to the protein of interest.

Considering the limitations of existing methods, effective and efficient methods for identification and quantification of dimer species was developed.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included.

In some exemplary embodiments, the disclosure provides a method identifying at least one product-related variant in a sample comprising a protein of interest.

As used herein, the term “protein” or “protein of interest” includes any amino acid polymer having covalently linked amide bonds. Proteins comprise one or more amino acid polymer chains, generally known in the art as “polypeptides”. “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. “Synthetic peptides or polypeptides' refers to a non-naturally occurring peptide or polypeptide. Synthetic peptides or polypeptides can be synthesized, for example, using an automated polypeptide synthesizer. Various solid phase peptide synthesis methods are known. A protein may contain one or multiple polypeptides to form a single functioning biomolecule. A protein can include any of biotherapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. In another exemplary aspect, a protein can include antibody fragments, nanobodies, recombinant antibody chimeras, cytokines, chemokines, peptide hormones, and the like. Proteins may be produced using recombinant cell-based production systems, such as the insect bacculovirus system, yeast systems (e.g., Pichia sp.), mammalian systems (e.g., CHO cells and CHO derivatives like CHO-K1 cells). For a review discussing biotherapeutic proteins and their production, see Ghaderi et al., “Production platforms for biotherapeutic glycoproteins. Occurrence, impact, and challenges of non-human sialylation,” (BIOTECHNOL. GENET. ENG. REV. 147-175 (2012)). In some exemplary embodiments, proteins comprise modifications, adducts, and other covalently linked moieties. Those modifications, adducts and moieties include for example avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as, globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, the protein can be an antibody, a bispecific antibody, a multispecific antibody, antibody fragment, monoclonal antibody, or an Fc fusion protein.

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H2} and C_{H}3. Each light chain comprises a light chain variable region (abbreviated herein as LCVR or V_{L}) and a light chain constant region. The light chain constant region comprises one domain (Cu). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different exemplary embodiments, the FRs of the anti-big-ET-1 antibody (or antigen-binding portion thereof) may be identical to the human germline sequences, or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a Fc fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. An antibody fragment may be produced by various means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains that are linked together, for example, by disulfide linkages. An antibody fragment may optionally comprise a multi-molecular complex.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

The term “Fc fusion proteins” as used herein includes part or all of two or more proteins, one of which is an Fc portion of an immunoglobulin molecule, that are not fused in their natural state. Preparation of fusion proteins comprising certain heterologous polypeptides fused to various portions of antibody-derived polypeptides (including the Fc domain) has been described, e.g., by Ashkenazi et al., Proc. Natl. Acad. ScL USA 88: 10535, 1991; Byrn et al., Nature 344:677, 1990; and Hollenbaugh et al., “Construction of Immunoglobulin Fusion Proteins”, in Current Protocols in Immunology, Suppl. 4, pages 10.19.1-10.19.11, 1992. “Receptor Fc fusion proteins” comprise one or more of one or more extracellular domain(s) of a receptor coupled to an Fc moiety, which in some embodiments comprises a hinge region followed by a CH2 and CH3 domain of an immunoglobulin. In some embodiments, the Fc-fusion protein contains two or more distinct receptor chains that bind to a single or more than one ligand(s). For example, an Fc-fusion protein is a trap, such as for example an IL-1 trap (e.g., Rilonacept, which contains the IL-1 RAcP ligand binding region fused to the IL-1R1 extracellular region fused to Fc of hIgG1; see U.S. Pat. No. 6,927,004, which is herein incorporated by reference in its entirety), or a VEGF Trap (e.g., Aflibercept, which contains the Ig domain 2 of the VEGF receptor Flt1 fused to the Ig domain 3 of the VEGF receptor Flk1 fused to Fc of hIgG1; e.g., SEQ ID NO:1; see U.S. Pat. Nos. 7,087,411 and 7,279,159, which are herein incorporated by reference in their entirety).

As used herein, the term “target” refers to any molecule that may specifically interact with a therapeutic protein in order to achieve a pharmacological effect. For example, the target of an antibody may be an antigen against which it is directed; the target of a ligand may be a receptor to which it preferentially binds, and vice versa; the target of an enzyme may be a substrate to which it preferentially binds; and so forth. A single therapeutic protein may have more than one target. A variety of targets are suitable for use in the method of the invention, according to the specific application. A target may, for example, be present on a cell surface, may be soluble, may be cytosolic, or may be immobilized on a solid surface. A target may be recombinant protein. In some exemplary embodiments, the target may be an antigen.

As used herein, the term “impurity” can include any undesirable protein present in the protein biopharmaceutical product. Impurity can include process and product-related impurities. The impurity can further be of known structure, partially characterized, or unidentified.

Process-related impurities can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

Product-related impurities (e.g., precursors, certain degradation products) can be molecular variants arising during manufacture and/or storage that do not have properties comparable to those of the desired product with respect to activity, efficacy, and safety. Such variants may need considerable effort in isolation and characterization in order to identify the type of modification(s). Product-related impurities can include truncated forms, modified forms, and aggregates. Truncated forms are formed by hydrolytic enzymes or chemicals which catalyze the cleavage of peptide bonds. Modified forms include, but are not limited to, deamidated, isomerized, mismatched S—S linked, oxidized, or altered conjugated forms (e.g., glycosylation, phosphorylation). Modified forms can also include any post-translationally modified form. Aggregates include dimers and higher multiples of the desired product. (Q6B Specifications: Test Procedures and Acceptance Criteria for Biotechnological/Biological Products, ICH August 1999, U.S. Dept. of Health and Humans Services).

Some product-related impurities or product-related protein variants have compromised binding affinity. Compromised binding affinity, here, includes a reduced binding affinity to the target of the protein of interest in the body or an antigen designed for the protein of interest. The compromised binding affinity can be any affinity which is less than the affinity of the protein of interest towards the target of the protein of interest in the body or an antigen designed for the protein of interest.

As used herein, the general term “post-translational modifications” or “PTMs” refers to covalent modifications that polypeptides undergo, either during (co-translational modification) or after (post-translational modification) their ribosomal synthesis. PTMs are generally introduced by specific enzymes or enzyme pathways. Many occur at the site of a specific characteristic protein sequence (signature sequence) within the protein backbone. Several hundred PTMs have been recorded, and these modifications invariably influence some aspect of a protein's structure or function (Walsh, G. “Proteins” (2014) second edition, published by Wiley and Sons, Ltd., ISBN: 9780470669853). The various post-translational modifications include, but are not limited to, cleavage, N-terminal extensions, protein degradation, acylation of the N-terminus, biotinylation (acylation of lysine residues with a biotin), amidation of the C-terminal, glycosylation, iodination, covalent attachment of prosthetic groups, acetylation (the addition of an acetyl group, usually at the N-terminus of the protein), alkylation (the addition of an alkyl group (e.g. methyl, ethyl, propyl) usually at lysine or arginine residues), methylation, adenylation, ADP-ribosylation, covalent cross links within, or between, polypeptide chains, sulfonation, prenylation, Vitamin C dependent modifications (proline and lysine hydroxylations and carboxy terminal amidation), Vitamin K dependent modification wherein Vitamin K is a cofactor in the carboxylation of glutamic acid residues resulting in the formation of a γ-carboxyglutamate (a glu residue), glutamylation (covalent linkage of glutamic acid residues), glycylation (covalent linkage glycine residues), glycosylation (addition of a glycosyl group to either asparagine, hydroxylysine, serine, or threonine, resulting in a glycoprotein), isoprenylation (addition of an isoprenoid group such as farnesol and geranylgeraniol), lipoylation (attachment of a lipoate functionality), phosphopantetheinylation (addition of a 4′-phosphopantetheinyl moiety from coenzyme A, as in fatty acid, polyketide, non-ribosomal peptide and leucine biosynthesis), phosphorylation (addition of a phosphate group, usually to serine, tyrosine, threonine or histidine), and sulfation (addition of a sulfate group, usually to a tyrosine residue). The post-translational modifications that change the chemical nature of amino acids include, but are not limited to, citrullination (the conversion of arginine to citrulline by deimination), and deamidation (the conversion of glutamine to glutamic acid or asparagine to aspartic acid). The post-translational modifications that involve structural changes include, but are not limited to, formation of disulfide bridges (covalent linkage of two cysteine amino acids) and proteolytic cleavage (cleavage of a protein at a peptide bond). Certain post-translational modifications involve the addition of other proteins or peptides, such as ISGylation (covalent linkage to the ISG15 protein (Interferon-Stimulated Gene)), SUMOylation (covalent linkage to the SUMO protein (Small Ubiquitin-related MOdifier)) and ubiquitination (covalent linkage to the protein ubiquitin). See European Bioinformatics Institute Protein Information ResourceSIB Swiss Institute of Bioinformatics, EUROPEAN BIOINFORMATICS INSTITUTE DRS—DROSOMYCIN PRECURSOR—Drosophila Melanogaster (FRUIT FLY)—DRS GENE & PROTEIN, http://www.uniprot.org/docs/ptmlist (last visited Jan. 15, 2019) for a more detailed controlled vocabulary of PTMs curated by UniProt.

As used herein, the term “chromatography” refers to a process in which a chemical mixture carried by a liquid or gas can be separated into components as a result of differential distribution of the chemical entities as they flow around or over a stationary liquid or solid phase. Non-limiting examples of chromatography include traditional reversed-phased (RP), ion exchange (IEX), mixed mode chromatography and normal phase chromatography (NP).

As used herein, the term “cation exchange chromatography” means a chromatography method which uses a “cation exchange chromatography material”. Further depending on the nature of the charged group the “cation exchange chromatography material” is referred to as e.g. in the case of cation exchange chromatography materials with sulfonic acid groups (S), or carboxymethyl groups (CM). Depending on the chemical nature of the charged group the “cation exchange chromatography material” can additionally be classified as strong or weak ion exchange chromatography material, depending on the strength of the covalently bound charged substituent. For example, strong cation exchange chromatography materials have a sulfonic acid group as chromatographic functional group.

For example, “cation exchange chromatography materials”, for example, are available under different names from a multitude of companies such as e.g. Bio-Rex, Macro-Prep CM (available from BioRad Laboratories, Hercules, Calif., USA), weak cation exchanger WCX 2 (available from Ciphergen, Fremont, Calif., USA), Dowex MAC-3 (available from Dow chemical company, Midland, Mich., USA), Mustang C (available from Pall Corporation, East Hills, N.Y., USA), Cellulose CM-23, CM-32, CM-52, hyper-D, and partisphere (available from Whatman plc, Brentford, UK), Amberlite IRC 76, IRC 747, IRC 748, GT 73 (available from Tosoh Bioscience GmbH, Stuttgart, Germany), CM 1500, CM 3000 (available from BioChrom Labs, Terre Haute, Ind., USA), and CM-Sepharose Fast Flow (available from GE Healthcare, Life Sciences, Germany). In addition, commercially available cation exchange resins further include carboxymethyl-cellulose, Bakerbond ABX, sulphopropyl (SP) immobilized on agarose (e.g. SP-Sepharose Fast Flow or SP-Sepharose High Performance, available from GE Healthcare—Amersham Biosciences Europe GmbH, Freiburg, Germany) and sulphonyl immobilized on agarose (e.g. S-Sepharose Fast Flow available from GE Healthcare, Life Sciences, Germany).

The “cation exchange chromatography materials” include mixed-mode chromatography materials performing a combination of ion exchange and hydrophobic interaction technologies (e.g., Capto adhere, Capto MMC, MEP HyperCell, Eshmuno HCX, etc.), mixed-mode chromatography material s performing a combination of anion exchange and cation exchange technologies (e.g., hydroxyapatite, ceramic hydroxyapatite, etc.), and the like. Cation exchange chromatography materials that may be used in cation exchange chromatography in the present invention may include, but are not limited to, all the commercially available cation exchange chromatography materials as described above. In an example of the present invention YMC BioPro SP-F column was used as cation exchange chromatography material.

As used herein, the term “mass spectrometer” includes a device capable of identifying specific molecular species and measuring their accurate masses. The term is meant to include any molecular detector into which a polypeptide or peptide may be eluted for detection and/or characterization. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization). The choice of ion source depends heavily on the application.

In some embodiments, the mass spectrometer can be an electrospray-mass spectrometer.

As used herein, the term “electrospray ionization” or “ESI” refers to the process of spray ionization in which either cations or anions in solution are transferred to the gas phase via formation and desolvation at atmospheric pressure of a stream of highly charged droplets that result from applying a potential difference between the tip of the electrospray needle containing the solution and a counter electrode. There are generally three major steps in the production of gas-phase ions from electrolyte ions in solution. These are: (a) production of charged droplets at the ES infusion tip; (b) shrinkage of charged droplets by solvent evaporation and repeated droplet disintegrations leading to small highly charged droplets capable of producing gas-phase ions; and (c) the mechanism by which gas-phase ions are produced from very small and highly charged droplets. Stages (a)-(c) generally occur in the atmospheric pressure region of the apparatus.

As used herein, the term “electrospray infusion setup” refers to an electrospray ionization system that is compatible with a mass spectrometer used for mass analysis of protein. In electrospray ionization, an electrospray needle has its orifice positioned close to the entrance orifice of a spectrometer. A sample, containing the protein of interest, can be pumped through the syringe needle. An electric potential between the syringe needle orifice and an orifice leading to the mass analyzer forms a spray (“electrospray”) of the solution. The electrospray can be carried out at atmospheric pressure and provides highly charged droplets of the solution. The electrospray infusion setup can include an electrospray emitter, nebulization gas, and/or an ESI power supply. The setup can optionally be automated to carry out sample aspiration, sample dispensing, sample delivery, and/or for spraying the sample.

In some exemplary embodiments, the electrospray ionization mass spectrometer can be a nano-electrospray ionization mass spectrometer.

The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

As used herein, the term “mass analyzer” includes a device that can separate species, that is, atoms, molecules, or clusters, according to their mass. Non-limiting examples of mass analyzers that could be employed for fast protein sequencing are time-of-flight (TOF), magnetic/electric sector, quadrupole mass filter (Q), quadrupole ion trap (QIT), orbitrap, Fourier transform ion cyclotron resonance (FTICR), and also the technique of accelerator mass spectrometry (AMS).

In some exemplary embodiments, mass spectrometry can be performed under native conditions.

As used herein, the term “native conditions” or “native MS” or “native ESI-MS” can include a performing mass spectrometry under conditions that preserve no-covalent interactions in an analyte. For detailed review on native MS, refer to the review: Elisabetta Boeri Erba & Carlo Petosa, The emerging role of native mass spectrometry in characterizing the structure and dynamics of macromolecular complexes, 24 PROTEIN SCIENCE 1176-1192 (2015). Some of the distinctions between native ESI and regular ESI are illustrated in table 1 and FIG. 1 (Hao Zhang et al., Native mass spectrometry of photosynthetic pigment-protein complexes, 587 FEBS Letters 1012-1020 (2013)).

In some exemplary embodiments, the mass spectrometer can be a tandem mass spectrometer.

As used herein, the term “tandem mass spectrometry” includes a technique where structural information on sample molecules is obtained by using multiple stages of mass selection and mass separation. A prerequisite is that the sample molecules can be transferred into gas phase and ionized intact and that they can be induced to fall apart in some predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on as long as one can obtain meaningful information or the fragment ion signal is detectable. Tandem MS have been successfully performed with a wide variety of analyzer combinations. What analyzers to combine for a certain application is determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time mass spectrometer ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization can include, but is not limited, to sequencing amino acids of the protein fragments, determining protein sequencing, determining protein de novo sequencing, locating post-translational modifications, or identifying post translational modifications, or comparability analysis, or combinations thereof.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com//proteinpilot), Phenyx (www.phenyx-ms.com), Sorcerer (www.sagenresearch.com), OMS SA (www.pubchem.ncbi.nlm.nih.gov/omssa/), X!Tandem (www.thegpm.org/TANDEM/), Protein Prospector (prospector.ucsfedu/prospector/mshome.htm), Byonic (www.proteinmetrics.com/products/byonic) or Sequest (fields.scripps.edu/sequest).

In some embodiments, the method for identifying at least one product-related variant can comprise using a competitive binding assay with insufficient antigen immobilized on a solid surface.

As used herein, the term “solid surface” can include any surface with an ability to bind to an antigen. Non-limiting examples of solid surface can include affinity resins, beads and coated plates with an immobilized protein, such as, avidin, streptavidin, or NeutrAvidin.

In some embodiments, the sample comprising the protein of interest can be digested after the competitive binding assay but prior to assessing it through SCX-MS.

In some embodiments, the sample comprising the protein of interest can be treated by adding a reducing agent to the sample.

As used herein, the term “reducing” refers to the reduction of disulfide bridges in a protein. Non-limiting examples of the reducing agents used to reduce the protein are dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof. In some specific embodiments, the treatment can further include alkylation. In some other specific exemplary embodiments, the treatment can include alkylation of sulfhydryl groups on a protein.

As used herein, the term “treating” or “isotopically labeling” can refer to chemical labeling a protein. Non-limiting examples of methods to chemically label a protein include Isobaric tags for relative and absolute quantitation (iTRAQ) using reagents, such as 4-plex, 6-plex, and 8-plex; reductive demethylation of amines, carbamylation of amines, ¹⁸O-labeling on the C-terminus of the protein, or any amine- or sulfhydryl-group of the protein to label amines or sulfhydryl group.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “hydrolyzing agent” refers to any one or combination of a large number of different agents that can perform digestion of a protein. Non-limiting examples of hydrolyzing agents that can carry out enzymatic digestion include trypsin, endoproteinase Arg-C, endoproteinase Asp-N, endoproteinase Glu-C, outer membrane protease T (OmpT), immunoglobulin-degrading enzyme of Streptococcus pyogenes (IdeS), chymotrypsin, pepsin, thermolysin, papain, pronase, and protease from Aspergillus Saitoi. Non-limiting examples of hydrolyzing agents that can carry out non-enzymatic digestion include the use of high temperature, microwave, ultrasound, high pressure, infrared, solvents (non-limiting examples are ethanol and acetonitrile), immobilized enzyme digestion (IMER), magnetic particle immobilized enzymes, and on-chip immobilized enzymes. For a recent review discussing the available techniques for protein digestion see Switazar et al., “Protein Digestion: An Overview of the Available Techniques and Recent Developments” (J. Proteome Research 2013, 12, 1067-1077). One or a combination of hydrolyzing agents can cleave peptide bonds in a protein or polypeptide, in a sequence-specific manner, generating a predictable collection of shorter peptides.

### Exemplary Embodiments

Embodiments disclosed herein provide methods for identifying at least one product-related variant in a sample comprising a protein of interest.

In some exemplary embodiments, this disclosure provides a method for identifying at least one product-related variant in a sample comprising a protein of interest, contacting a sample including a protein of interest and at least one product-related variant to a competitive binding condition, wherein a binding condition provides an insufficient antigen immobilized on beads and wherein said at least one product-related variant has compromised binding with said insufficient antigen; incubating said sample with said insufficient antigen; collecting a flow-through from washing after incubating; and identifying the at least one product-related critical quality attributes in said flow-through using a liquid chromatography-mass spectrometer.

In some exemplary embodiments, a product-related variant is one or more of truncated forms, modified forms, and aggregates of the protein of interest.

In some exemplary embodiments, a product-related variant is deamidated, isomerized, mismatched S—S linked, oxidized, and/or altered conjugated form (e.g., glycosylation, phosphorylation) of the protein of interest.

In some exemplary embodiments, a product-related variant is a post-translationally modified form.

In some exemplary embodiments, a product-related variant has a compromised binding affinity, wherein the compromised binding affinity is about 90% the binding affinity of the protein of interest, about 80% the binding affinity of the protein of interest, about 70% the binding affinity of the protein of interest, about 60% the binding affinity of the protein of interest, about 50% the binding affinity of the protein of interest, about 40% the binding affinity of the protein of interest, about 30% the binding affinity of the protein of interest, about 20% the binding affinity of the protein of interest, or is about 10% the binding affinity of the protein of interest.

In some exemplary embodiments, the mass spectrometer can be a nano-electrospray ionization mass spectrometer.

In some exemplary embodiments, the electrospray ionization mass spectrometer can be run under native conditions.

It is understood that the methods are not limited to any of the aforesaid protein, impurity, and column and that the methods for identifying or quantifying may be conducted by any suitable means.

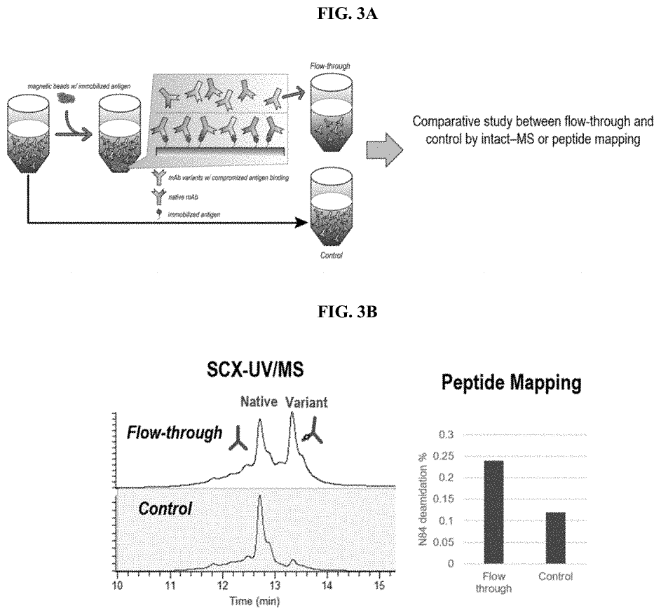

An exemplary embodiment is illustrated in FIGS. 3A and 3B. To a sample comprising the protein if interest and possibly its variants, beads with immobilized antigen can be added. The amount of beads with immobilized antigen is such that not all the protein of interest (native mAb) and its variants can bind to it. Any variant with a reduced binding affinity to the antigen will have lower chance to bind due to the limited amount of antigen present. The flow-through (unbound fraction) can be collected and analyzed using SCX-MS or peptide mapping. The control (i.e., the sample without the immobilized antigen binding assay step) can also be analyzed using SCX-MS or peptide mapping. The comparative study between the flow-through and control can lead to a chromatogram as illustrated in FIG. 3B. Any variant with reduced binding affinity would be more abundant in the flow-through. On comparison of the amount of the variant as such identified, it can be seen that the relative percentage of the variant is more in the flow-through than the control due to its reduced binding affinity.

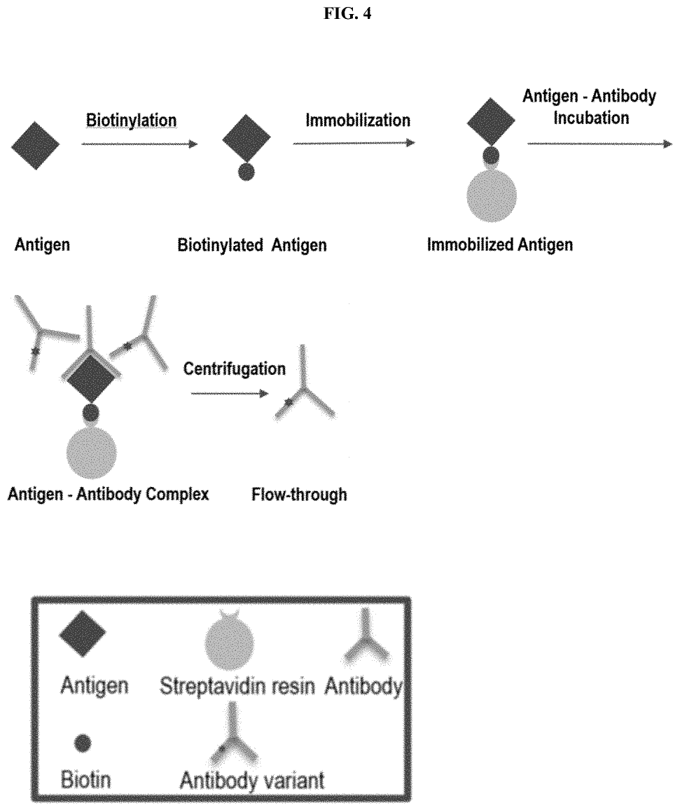

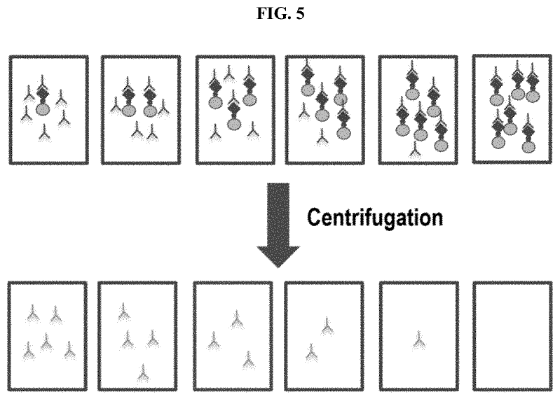

Such an experiment can be devised using the workflow as shown in FIG. 4 and FIG. 5.

For the present invention, the ratio between the antigen and protein of interest is very important. The amount of the antigen added can be such that about 25% to about 75% of the protein of interest can bind to the antigen. In some embodiments, the amount of the antigen added can be such that about 50% of the protein of interest can bind to the antigen.

The consecutive labeling of method steps as provided herein with numbers and/or letters is not meant to limit the method or any embodiments thereof to the particular indicated order.

Various publications, including patents, patent applications, published patent applications, accession numbers, technical articles and scholarly articles are cited throughout the specification. Each of these cited references is herein incorporated by reference, in its entirety and for all purposes.

The disclosure will be more fully understood by reference to the following Examples, which are provided to describe the disclosure in greater detail. They are intended to illustrate examples and should not be construed as limiting the scope of the disclosure.

### EXAMPLES

Materials. Deionized water was provided by a Milli-Q integral water purification system installed with a MilliPak Express 20 filter (Millipore Sigma, Burlington, Mass.). mAb1, mAb2, mAb1 antigen, and mAb2 antigen were generated in-house at Regeneron (Tarrytown, N.Y.).

Online nSCX-UV/MS Analysis.

Strong cation exchange chromatography was performed using a YMC BioPro SP-F (YMC, Japan). For the sample separations, the mobile phases used were 20 mM Ammonium Acetate, pH 5.6 (Mobile phase A) and 150 mM Ammonium Acetate pH 7.4 (Mobile phase B). A linear pH gradient was used to elute charge variants of mAb1 with detection at 280 nm.

Prior to sample injection, the column compartment temperature was set at 45° C. and a strong cation exchange column (100 mm 4.6 mm, 5 μm) (YMC, Japan) was preconditioned with mobile phase A (20 mM ammonium acetate, pH adjusted to 5.6 with 20 mM acetic acid) at a flow rate of 0.4 mL/min. Upon the injection of an aliquot (10 μg) of the protein samples, the gradient is held at 100% mobile phase A for 2 minutes followed by a linear increase to 100% mobile phase B (150 mM ammonium acetate, pH 7.4) in 16 minutes. The gradient was held at 100% mobile phase B for 4 minutes and then returned to 100% mobile phase A to recondition the column for 7 minutes before the next injection. Peaks at a relative residence time earlier or later than the main peak are identified using an online MS.

For the mass spectrometric analysis, the resolution was set at 17,500, the capillary spray voltage was set at 1.5 kV, the in-source fragmentation energy was set at 100, the collision energy was set at 10, the capillary temperature was set at 350° C., the S-lens RF level was set at 200 and the HCD trapping gas pressure was set at 3. Mass spectra were acquired with an m/z range window between 2000 and 15000.

Data analysis. Protein Metrics Intact Mass software was used for raw data deconvolution. Thermo Xcalibur Qual Browser was used for extracted ion chromatogram analysis.

### Example 1

### 1.1 Optimization of the Competitive Binding Assay

A competitive binding assay was developed to differentiate variants of a protein of interest with impaired binding. mAb1 was used as an exemplary protein of interest.

mAb1 antigen was biotinylated using a biotinylation reagent (30 min at room temperature, using NHS-biotin). Biotinylated mAb1 was loaded onto a bed of streptavidin-resin (7 nmol biotin binding capacity, Pierce) in a tube (micro BioSpin, Bio-Rad). After five minutes, filtration was effected by centrifugation, and the gel bed was washed with 100 mM Tris, pH 7.5 (about 1 minute incubation, then spin) and then six times with purified water (Milli-Q, Millipore), to obtain antigen-immobilized resin.

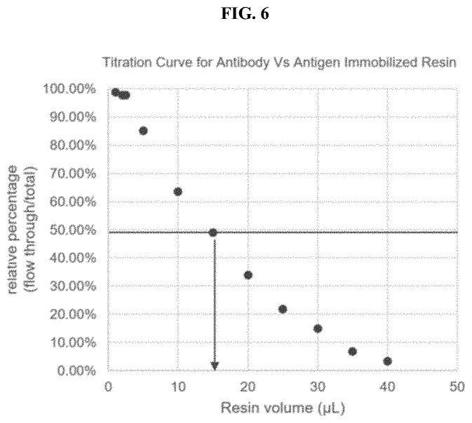

A series of tubes with eleven different increasing volumes of antigen-immobilized resin (1-40 μL) were suspended in binding assay buffer (binding assay buffer can be anything from Tris to PBS buffer). On addition of purified mAb1, the tubes were incubated for 1 hour at 4° C. On centrifugation i.e., spinning down at 800×g for five minutes at 4° C., the supernatant was removed and the binding was analyzed by measuring the protein concentration of the flowthrough at 280 nm using a NanoDrop UV-Vis spectrophotometer, relative to the total amount of mAb1. An exemplary embodiment of a titration curve obtained is shown in FIG. 6. The volume of antigen-immobilized resin was insufficient to capture all of the mAb1 sample, thus providing a flowthrough enriched for any binding-impaired variants of mAb1. The resin volume needed to obtain 50% binding of mAb1 was selected for further competitive binding assays.

### 1.2 Competitive Binding-nSCX-UV/MS Analysis.

A competitive binding assay as described above was performed for mAb1.

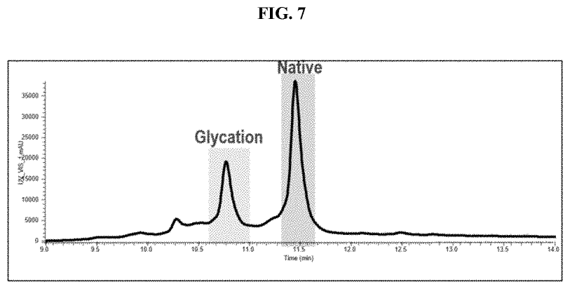

SCX-UV analysis of mAb1 shows that it features a substantial glycation variant, as shown in FIG. 7. The specific glycation site was identified as heavy chain (HC) lysine (K) 98, as shown in Table 1. This glycation has previously been implicated in antigen-binding, but its exact impact was unknown.

TABLE 1

| | | | PTM |
|---|---|---|---|
| | Site | Peptide | Percentage |
| PTM | Location^{a} | Sequence | (%) |
| Asparagine | HC Asn⁸⁴ | (K)NSLFLQMNSL | 1.1% |
| R(A) | | | |
| Deamidation | | | |
| | HC Asn³¹¹/ | (R)VVSVLTVLHQ | 2.5% |
| | HC* Asn³¹⁸ | DWLNGK(E) | |
| | HC Asn³⁸⁰/ | (K)GFYPSDIAVE | 4.0% |
| | HC*Asn³⁸⁷ | WESNGQPENNYK(T) | |
| Methionine | HC Met²⁴⁸/ | (K)DTLMISR(T) | 3.7% |
| Oxidation | HC* Met2⁵⁵ | | |
| | HC Met⁴²⁴ | (R)WQEGNVFSCSV | 1.2% |
| | | MHEALHNHYTQK(S) | |
| | HC* Met⁴³¹ | (R)WQEGNVFSCSV | 1.4% |
| | | MHEALHNR(F) | |
| Lysine | HC Lys⁹⁸ | (E)DTAVYFCV | 31.7% |
| Glycation | | K(D) | |
| Lysine | | | 4.3% |
| Glucuronylation | | | |
| Lysine | | | 1.0% |
| Carboxy- | | | |
| methylation | | | |
| Lysine +161.01 | | | 1.3% |
| Da Modification | | | |

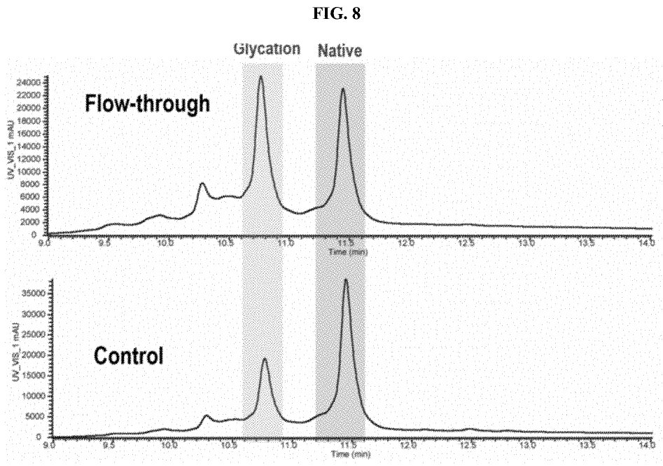

In order to determine whether any major variants of mAb1, such as glycation, impact antigen-binding, mAb1 flow through from the competitive binding assay was compared to a control sample of mAb1 using SCX-UV analysis. The control experiment included use of SCX-UV/MS on the mAb1 obtained from stability study without any enrichment step. The comparison of the two chromatograms is shown in FIG. 8. FIG. 8 clearly shows enrichment of the glycation peak of mAb1 in flow through from the competitive binding assay as compared to control mAb1. This demonstrates that the glycation modification indeed impairs binding of mAb1 to mAb1 antigen, and is therefore a CQA that should be taken into consideration in product development.

### 1.3 Evaluation of Multiple Critical Quality Attributes Using Competitive Binding-SCX-MS.

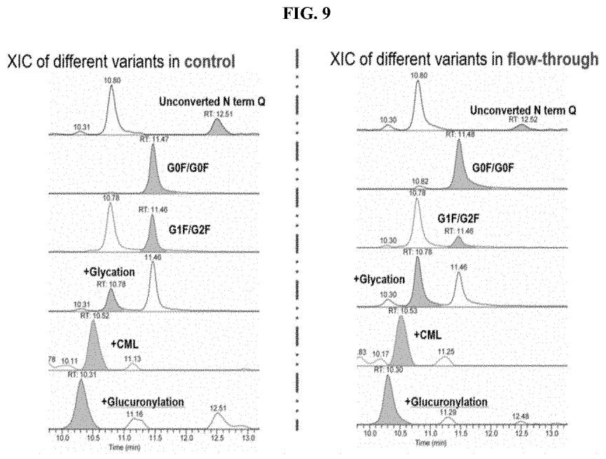

The samples from Example 1.2 were further subject to mass spectrometry analysis. Extracted-ion chromatograms (XIC) from the mAb1 control sample and the mAb1 flow through of the competitive binding assay are shown in FIG. 9. Several protein variants are identifiable in the compared XICs, demonstrating that the method of the invention is capable of simultaneously identifying several CQAs that adversely affect protein binding. At the same time, PTMs that have no change in relative abundance between the samples may be disregarded as CQAs.

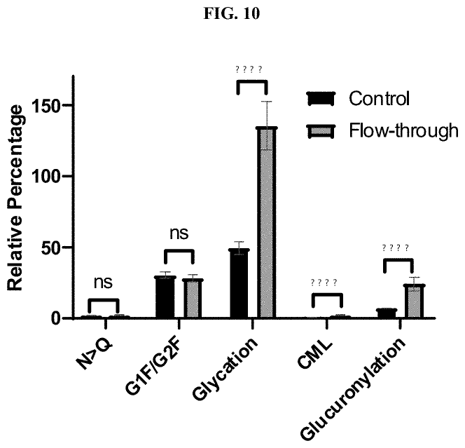

A statistical analysis of the enrichment of PTMs in the competitive binding assay flow through compared to the control sample is shown in FIG. 10. It is clear from the comparison that some modifications (such as HC K98 glycation, HC K98 carboxymethylation (CML), and HC K98 glucuronylation) were enriched using the competitive binding assay experiment, identifying them as CQAs for mAb1, while others (N-term Q and terminal galactosylation of Fc glycan) were not. Thus, the method successfully identified critical quality attributes and product-related variants that cause reduced binding of mAb1 to mAb1 antigen, and distinguished them from modifications that do not impact binding and thus can be disregarded in product development.

### Example 2

### 2.1 Competitive Binding-SCX-MS Analysis of a Bispecific Antibody

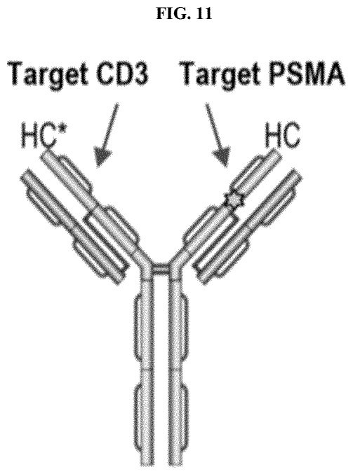

The effectiveness of the method of the invention was further demonstrated by analysis of a bispecific antibody, bsAb1. The structure of bsAb1 is shown in FIG. 11. FIG. 11 shows that mAb2 comprises two distinct HC regions, HC and HC*.

Previous nSCX-MS analysis of bsAb1 lots has shown that it prominently features a deamidation variant. Previous peptide mapping analysis has identified a major variant caused by deamidation at HC N56, as shown in Table 2.

TABLE 2

| | | | t0 in | | Platform, pH6, | |
|---|---|---|---|---|---|---|
| Site | Peptide | | Platform | | 45C28d | |
| Loca- | Se- | PTM | Mod | Total | Mod | Total |
| tion | quence | name | (%) | (%) | (%) | (%) |
| HC | QVQLVE | Q1 > | 94.2 | 94.2 | 99.6 | 99.6 |
| Gln1 | SGGGWQ | pyro- | | | | |
| | PGR | Glu | | | | |
| HC | LSCAAS | M34 | 0.2 | 0.2 | 0.2 | 0.2 |
| Met34 | GFTFSS | Oxida- | | | | |
| | YGMHWV | tion | | | | |
| | R | | | | | |
| HC | GLEWVA | N56 | 15.1 | 15.1 | 30.4 | 30.4 |
| Asn56 | VISYAG | Deamida- | | | | |
| | NNK | tion | | | | |
| HC | DSYYDF | D99 | 0.2 | 0.2 | 1.9 | 1.9 |
| Asp99 | LTDPDV | Dehydra- | | | | |
| | LDIWGQ | tion | | | | |
| | GTMVTV | | | | | |
| | SSASTK | | | | | |
| HC | DSYYDF | M119 | 3.4 | 3.4 | 9.3 | 9.3 |
| Met119 | DPLTDV | Oxida- | | | | |
| | GLDIWQ | tion | | | | |
| | GTMVTV | | | | | |
| | SSASTK | | | | | |
| HC | DTLMIS | M255 | 5.2 | 5.2 | 8.7 | 8.7 |
| Met255/ | R | Oxida- | | | | |
| HC* | | tion | | | | |
| Met255 | | | | | | |

HC N56 is located in the complementarity-determining region (CDR) of bsAb1, raising the possibility that it may adversely impact binding of bsAb1 to its target. In order to determine any potential impact of bsAb1 variants on binding, bsAb1 was subjected to competitive binding-SCX-MS analysis.

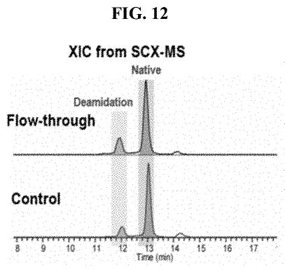

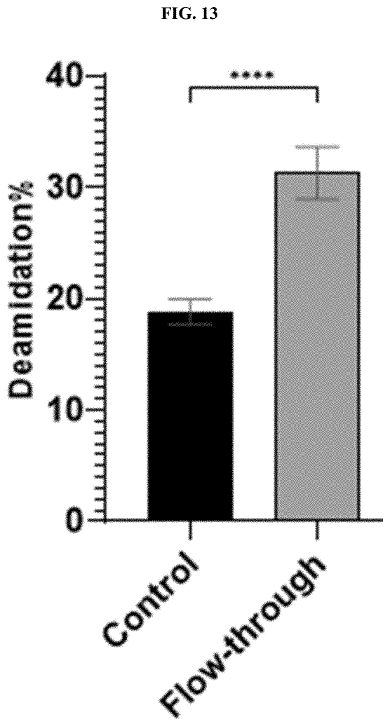

Antigen-immobilized resin was optimized and prepared as described in Example 1.1. bsAb1 was subjected to the competitive binding assay, and flow through from the competitive binding assay was compared to bsAb1 control sample using SCX-UV/MS. The comparison of the two UV chromatograms is shown in FIG. 12. FIG. 12 clearly shows enrichment of a deamidation variant of bsAb1 in the flow through from the competitive binding assay, which is quantified as shown in FIG. 13. The enrichment of a deamidation variant of bsAb1 in the flow through of the competitive binding assay demonstrates that deamidation is a CQA in the production of bsAb1.

### 2.2 Evaluation of Multiple Critical Quality Attributes Using Competitive Binding-SCX-MS.

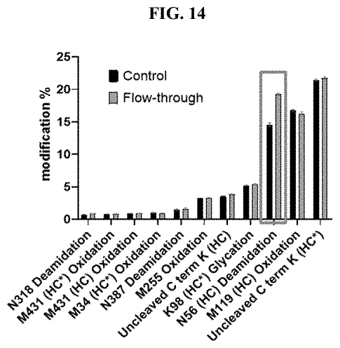

bsAb1 was subjected to further analysis using competitive binding-SCX-peptide mapping MS. The extracted-ion chromatograms (XIC) from the control experiment and the competitive binding assay flow through showed several different PTMs. A comparative analysis of the amount of variants obtained using the control experiment and the competitive binding assay experiment is shown in FIG. 14. It is clear from the comparison of the variants that only the N56 deamidation variant was enriched using the competitive binding assay experiment, and thus was probably the only identified critical quality attribute or product-related variant of bsAb1 with reduced binding affinity.

What is claimed is:

1. A method for characterizing at least one product-related variant, said method comprising:
a. obtaining a sample including a protein of interest and at least one product-related variant of said protein of interest;
b. contacting said sample to a competitive binding condition including an insufficient target immobilized on beads;
c. washing said beads to collect a flow-through;
d. subjecting said flow-through to liquid chromatography-mass spectrometry analysis to separate said protein of interest and said at least one product-related variant; and
e. comparing the abundance of said at least one product-related variant from (d) to an abundance of said at least one product-related variant obtained from a liquid chromatography-mass spectrometry analysis of a control sample of (a) to characterize said at least one product-related variant.

2. The method of claim 1, wherein the liquid chromatography is strong cation exchange chromatography.

3. The method of claim 1, wherein said beads are agarose beads or magnetic beads.

4. The method of claim 1, wherein said flow-through is enriched for said at least one product-related variant.

5. The method of claim 1, wherein said flow-through of (c) is collected by performing centrifugation.

6. The method of claim 1, further comprising subjecting said flow-through of (c) to digestion conditions prior to liquid chromatography-mass spectrometry analysis.

7. The method of claim 1, wherein said beads are coated with streptavidin resin.

8. The method of claim 1, wherein said insufficient target includes an amount of said target capable of binding to about 30% to about 80% of said protein of interest.

9. The method of claim 1, wherein said sample of (b) is incubated for about one hour.

10. The method of claim 1, wherein said sample of (b) is incubated at about room temperature.

11. The method of claim 1, wherein said product-related variant comprises a size-variant.

12. The method of claim 11, wherein said size-variant is a fragmentation variant of said protein of interest.

13. The method of claim 11, wherein said size-variant is an aggregation variant of said protein of interest.

14. The method of claim 1, wherein said product-related variant comprises a charge-variant of said protein of interest.

15. The method of claim 1, wherein said product-related variant comprises a post translationally modified-variant of said protein of interest.

16. The method of claim 1, wherein said target is an antigen directed to said protein of interest.

17. The method of claim 1, wherein said product-related variant is characterized as a critical quality attribute if said abundance of said at least one product-related variant from (d) is significantly more than said abundance of said at least one product-related variant obtained from the liquid chromatography-mass spectrometry analysis of the control sample of (a).
